(19) United States
(12) Patent Application Publication
(10) Pub. No.: US 2024/0082250 A1
(43) Pub. Date: Mar. 14, 2024

(54) NEDDYLATION INHIBITION FOR USE IN THE TREATMENT OF HUMAN IMMUNODEFICIENCY VIRUS (HIV) INFECTION

(71) Applicant: Northwestern University, Evanston, IL (US)

(72) Inventors: Richard Thomas D'Aquila, Evanston, IL (US); Chisu Song, Evanston, IL (US); Isabelle Clerc, Evanston, IL (US); Cristina Vaca, Evanston, IL (US); Aubrey Sawyer, Evanston, IL (US)

(21) Appl. No.: 18/467,698

(22) Filed: Sep. 14, 2023

Related U.S. Application Data
(60) Provisional application No. 63/375,653, filed on Sep. 14, 2022.

(51) Int. Cl. A61K 31/519; A61P 31/18

(52) U.S. Cl. CPC A61K 31/519; A61P 31/18

(57) ABSTRACT

Disclosed is a method for the treatment of a subject having an HIV infection or inhibiting neddylation using a neddylation inhibiting enzyme, neural-precursor-cell-expressed developmentally down-regulated 8 (NEDD8)-activating enzyme E1 (NAE1).

### CROSS REFERENCE TO RELATED APPLICATION

This application claims benefit of U.S. Provisional Application Ser. No. 63/375,653, filed Sep. 14, 2022, the entire contents of which is incorporated by reference herein.

### STATEMENT REGARDING FEDERALLY SPONSORED RESEARCH

This invention was made with government support under 5P01AI131346-05, 5P01AI131346-04, 1P01AI131346-01, P30AI117943, and T32AI007476 awarded by the National Institutes of Health. The government has certain rights in the invention.

### FIELD OF THE INVENTION

The disclosed technology is generally directed to methods for improving treatment of HIV infection and enabling prolonged remission after ending treatment (e.g., stopping all medications), and/or limiting HIV-associated comorbidities related to treatment. More particularly the technology is directed to the use of neddylation inhibition for improving treatment for HIV infection.

### BACKGROUND

Infection and treatment of human immunodeficiency virus (HIV) are an ongoing struggle. HIV can now be very well treated by easy-to-take medications (antiretroviral therapy, ART), but the virus persists in some T cells throughout the body during ART. These T cells either make just pieces of the virus (without making infectious viruses that can spread to other cells) or they harbor viruses that are completely quiescent and not making any virus components (HIV's latent reservoir); at least some of these cells in each person with HIV infection remain capable of making infectious virus. While effective suppression of ongoing virus replication blocks development of immunodeficiency (AIDS), it does not prevent ongoing uninfected immune cell activation and systemic inflammation from the immune recognition of virus or its remnants, which causes other associated diseases (e.g. cardiovascular disease, cancers not caused by immunodeficiency, other organ damage) to occur prematurely, and more aggressively, in ART-treated patients. Moreover, ART must be taken consistently lifelong as viremia returns to high levels that inflicts progressive damage to the immune system within a few weeks of stopping ART. Better treatment options for HIV are needed that are effective in preventing or limiting immune cell activation and systemic inflammation from HIV and/or preventing or markedly delaying viremia rebound after stopping ART.

It has been reported in the peer-reviewed literature that a neddylation inhibitor decreases infectivity of HIV produced after an acute infection of cells ex vivo by increasing virion content of APOBEC3G (A3G). However, there are no reports on effects of neddylation inhibition on reactivation of latent HIV proviruses or infectivity of reactivated viruses.

### SUMMARY

An aspect of the disclosure is methods of treating a human immunodeficiency virus (HIV) infection in a subject comprising administering to the subject an inhibitor of neddylation activation enzyme neural-precursor-cell-expressed developmentally down-regulated 8 (NEDD8) E1 (NAE1). Embodiments include methods of improving treatment of HIV infection by administering an inhibitor of NAE1 along with antiviral therapy (ART) or when stopping ART. Embodiments include use of an inhibitor of NAE1 improve treatment of HIV infection and decrease production of infectious HIV from latent HIV provirus or other HIV latency components.

Another aspect of the disclosure is directed to pharmaceutical composition that includes an inhibitor of NAE1 for use in the described methods.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates the role of A3G in HIV-1 infection (from: Harris R S, Hultquist J F, Evans D T. The restriction factors of human immunodeficiency virus. J Biol Chem. 2012; 287:40875-40883).

FIG. 2 shows that MLN inhibits neddylation at 100 mM and 200 mM. Immunoblot for Cullin 2 (CUL2) shows that MLN treatment of ACH2 cells reduces Cullin RING neddylation with and without reactivation via tumor necrosis factor (TNFa). Data not shown indicate similar effects of MLN on CUL2 neddylation in CEM, Jurkat and J-Lat cells.

FIG. 3 shows effects of inhibiting neddylation when reactivating HIV from latently infected ACH2 cells with 3 different latency reactivating agents (LRAs): TNFa at 10 ng/mL; PMA/ionomycin (PMAi) at 405 pM phorbol 12-myristate 13-acetate (PMA) and 6.7 nM ionomycin (eBiosciences™ stimulation cocktail); and JQ1 at 100 nM, compared to neddylation inhibitor-untreated ACH2 cells also activated by these LRAs, on: a) the infectivity of HIV in culture supernatant fluid (Panels 1-4); b) amount of A3G incorporated into virions in culture supernatant fluid (Panels 1 and 2). (A) HIV produced from ACH2 cells treated with MLN before and during LRA exposure was less infectious compared to virus produced from untreated cells (DMSO) in multiple separate experiments using each of the 3 LRAs. Panel 1 in A shows TNFa reactivation with MLN 200 nM, n=5 separate ACH2 cultures; Panel 2 in A shows a separate set of experiment with MLN at 100 nM and 200 nM, n=2 separate ACH2 cultures at each concentration; Panel 3 in A shows PMAi reactivation with MLN at 100 nM and 200 nM, n=5 separate ACH2 cultures at each concentration; Panel 4 in A. shows JQ1 reactivation with MLN at 100 nM and 200 nM, n=4 separate ACH2 cultures at each concentration. (B) MLN treatment of ACH2 cells before and during TNFa provirus reactivation increases the amount of A3G incorporated into virions, a strong candidate mechanism for decreased infectivity shown in (A). Virions from ACH2 cell supernatant were pelleted over a 20% sucrose cushion. Immunoblotting of viral pellets reveals A3G inside the virions is increased with MLN treatment (Panel 1 in B shows one representative image). A3G signal (black bars) and p24 signal (grey bars) from n=3 experiments are quantified on the right (Panel 2 in B).

FIG. 4 shows that MLN (200 nM) alters intracellular distribution of A3G to increase proportion in low molecular mass (LMM) form from which it is packaged into budding virions rather than non-packageable high molecular mass (HMM) complexes. A3G low molecular mass forms (LMM) (fractions 1-5) and HMM complexes (fractions 7-12) are visualized by immunoblotting of fractions collected after density gradient centrifugation of a 5-40% sucrose gradient. (A) Treatment of H9 cells with 200 nM MLN for 4 or 6 days before density gradient centrifugation and immunoblotting of gradient fractions shows shift to LMM form. (B) CD4+CD45RO+ memory T cells were isolated from healthy donors; cells were then stimulated with CD3/28 Abs (*) and cultured for 4 days with DMSO, 100 nM, or 200 nM MLN before density gradient centrifugation and immunoblotting of gradient fractions. This again shows a shift to LMM form.

FIG. 5 shows effects of inhibiting neddylation on amount of HIV capsid antigen (p24) present in ACH2 cell culture supernatant fluid after provirus reactivation by TNFa, PMAi and JQ1, as well as in virions reactivated by TNFa. (A) Extracellular p24 antigen in culture supernatant fluid as measured via ELISA is significantly reduced when ACH2 cells are treated with MLN before and during exposure to LRAs, n=3 for each: TNFa 10 ng/mL in Panel 1; PMA/i in Panel 2; JQ1 in Panel 3. (B) Immunoblotting of reactivated virions pelleted from culture supernatant fluid from LRA-exposed ACH2 and J-Lat 6.3 cells shows reduced p24 antigen with MLN treatment compared to control.

FIG. 6 shows MLN reduces intracellular p24 production from: a) ACH2 exposed to three LRAs (TNFa, PMA/i, JQ1); b) a second latently infected T cell line (J-Lat 6.3) after exposure to TNFa; and c) decreases HIV promoter-driven GFP expression from both J-Lat 6.3 and J-Lat 11.1 lines exposed the three LRAs. (A) MLN significantly reduces intracellular p24 production from ACH2 treated with TNFa (n=4), PMA/i (n=4,), and JQ1 (n=3), compared to DMSO control. (B) MLN reduces intracellular p24 production from J-Lat 6.3 cells exposed to TNFa, as well as from ACH2 cells exposed to TNFa. J-Lat cell lines do not express A3G, contain an incomplete HIV provirus that does not produce infectious viruses (and does express GFP from the HIV promoter), in contrast to ACH cells that contain A3G; an intact, reporter-free HIV genome; and can produce infectious viruses. (C) Six hour treatment of J-Lat cells (both J-Lat 6.3 and J-Lat 11.1) with MLN before and during LRA exposure reduces HIV promoter-driven GFP expression after exposure to TNFa, PMAi and JQ1.

FIG. 7 shows HIV gag RNA expression that increases after ACH2 cells are exposed to either TNFa or PMAi is blunted with MLN treatment, leading to a decrease in proviral transcriptional activity after these LRAs relative to DMSO. Gag RNA is reduced in ACH2 cells as measured by RT-qPCR 48 hours after reactivation. ACH2 cells were treated with either DMSO or MLN (at 100 mM or 200 mM) before and during exposure to either 10 ng/mL TNFa (plot on left) or 405 pM PMA and 6.7 nM ionomycin (eBiosciences stimulation cocktail) (plot on right); 4 separate ACH2 cell cultures were used for each plot's experiment.

FIG. 8 shows MLN induces apoptotic cell death in reactivated latently infected ACH 2 T cells and TCR-activated, uninfected primary CD4+ T cells. (A) Annexin V staining of ACH2 cells shows an increase in apoptosis in MLN treated cells. ACH2 cells were pretreated for 6 hours with 200 nM MLN prior to provirus reactivation with 10 ng/mL TNFa. 16 hours post-TNFa addition cells were collected; apoptosis was measured via flow cytometry by staining for Annexin V and SYTOX Green DNA intercalant. Plot shows that percentage of cells dying specifically from apoptosis increases with MLN treatment before/during TNFa exposure. (B) RT-qPCR for apoptotic targets (PUMA, NOXA) and proliferation/cell cycle indicators (p21, HIF1a) shows an increase in pro-apoptotic gene expression in ACH2 cells. RT-qPCR shows increased relative expression of RNAs encoding NOXA, PUMA, and p21 in ACH2 cells treated with 200 nM MLN before and during exposure to 10 ng/mL TNFa. p53 RNA does not increase with MLN in ACH2 cells. Data was collected 24 hours after TNFa addition. (C) CD4+ T cells from 3 healthy donors were activated for 48 hrs with anti-CD3/CD28 antibodies. Cells were then treated with 200 nM of MLN and apoptosis was measured after 6, 16, and 24 hrs of drug treatment using flow cytometry. * p<0.05 *** p=0.0002 (D) Expression of RNA encoding cell cycling and apoptotic targets assessed before in ACH2 cells was determined in primary CD4 T cells via RT-qPCR. Gene expression was normalized to 18S control ** p=0.0033 **** p<0.0001 A&B) n=3 donors.

### DETAILED DESCRIPTION

Disclosed are new strategies that can contribute to achieving a “functional cure” of HIV infection (sustained suppression of HIV viremia after stopping ART) via several different mechanisms. For the first time, it is shown that an inhibitor of a neddylation-activating enzyme, neural-precursor-cell-expressed developmentally down-regulated 8 (NEDD8) E1 (NAE1), e.g., pevonedistat (formerly MLN4924, and called MLN here), decreases production of components of human immunodeficiency virus (HIV) after HIV provirus transcription-triggering stimuli are received by latently infected T cell lines harboring a quiescent HIV genome (such reversible virus latency occurs in people and is a key reason why lifelong antiretroviral therapy (ART) is mandated by current guidelines). Aspects of this disclosure are methods of improving treatment of HIV infection by administering an inhibitor of NAE1 along with ART or when stopping ART. Aspects include methods of treating a HIV infection in a subject by administering to the subject a NAE1 inhibitor. In embodiments, the inhibitor of NAE1 is pevonedistat (or MLN, as used interchangeably in this disclosure).

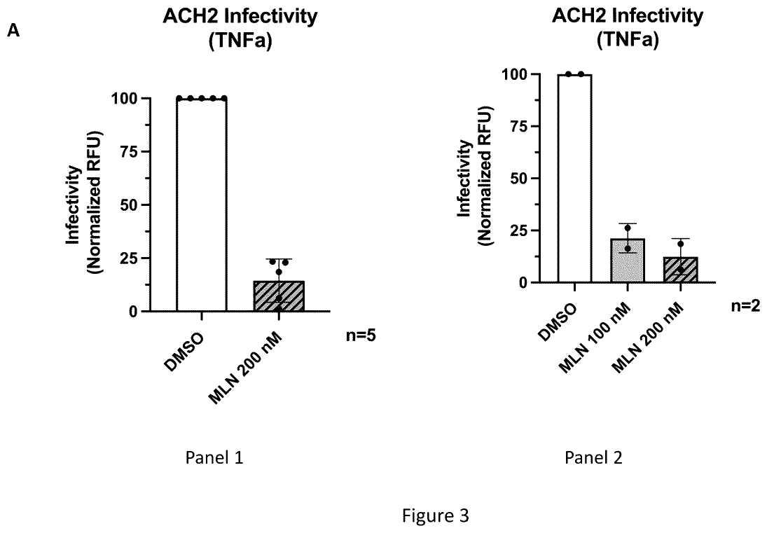

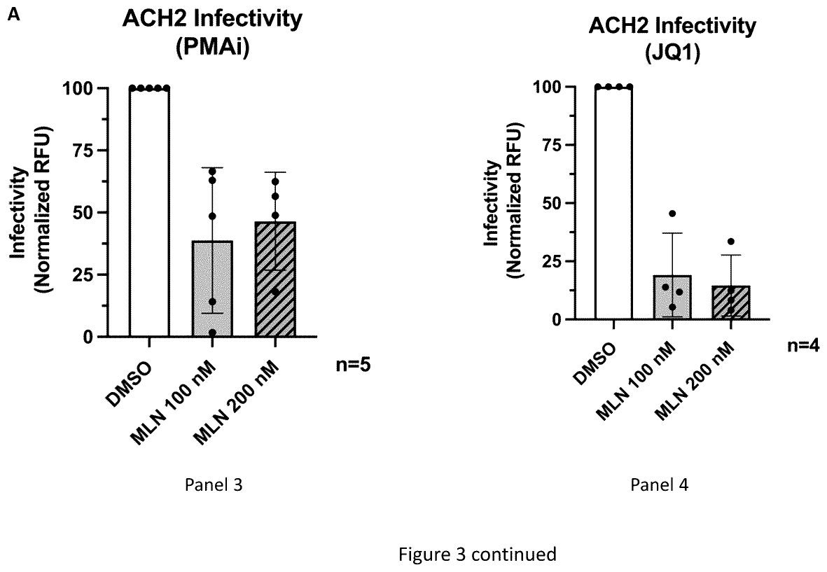

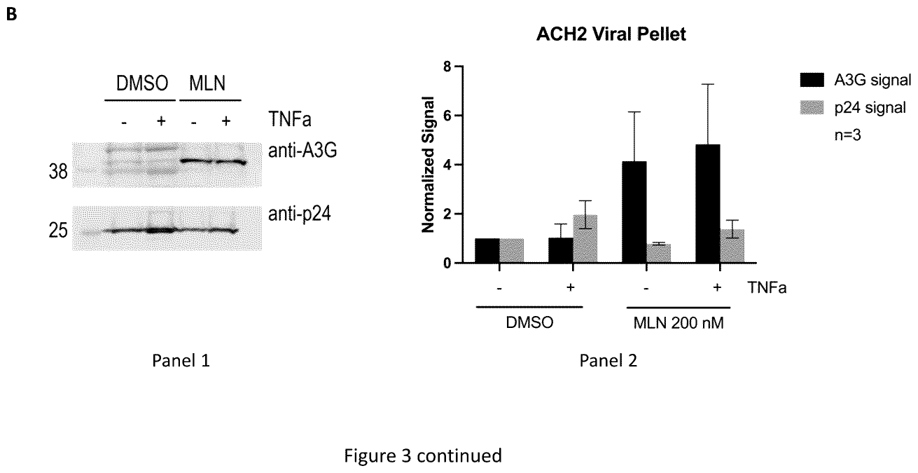

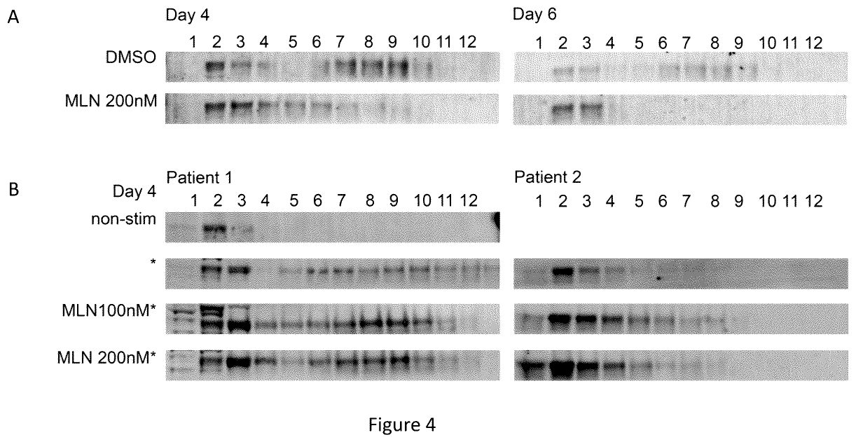

Apolipoprotein B mRNA-editing, catalytic polypeptide-like 3 (APOBEC3G, or A3G) is one of seven human APOBEC3 cytidine-deaminases. A3G strongly inhibits HIV-1 replication by mutating C to T in HIV ssDNA during reverse transcription if packaged into virions. The inhibitor of NAE1 is identified to decrease reactivated virion infectivity and increase reactivated virion packaged A3G following provirus transcription-triggering stimuli (FIG. 3). In addition, NAE1 inhibition limits the cellular sequestration of anti-HIV cytoplasmic APOBEC3 cytidine deaminases into high molecular mass (HMM) ribonucleoprotein complexes from which packaging into a self-assembling virion cannot occur (FIG. 4). This cellular sequestration contributes, along with HIV-mediated degradation of cytoplasmic APOBEC3s by a specific Cullin-RING ligase (CRL) including an HIV-encoded substrate adapter (virion infectivity factor, Vif), to exclusion of these antiviral gene editors from virions that self-assemble after HIV reactivates from latency. Exclusion of these factors that block viral reverse transcription and integration from virions is necessary for the infection to spread to uninfected cells and eventually lead to viremia. Earlier work also demonstrated that pevonedistat's blockade of neddylation of all cullins prevents the ubiquitylating activity of the specific CRL (CRL^{Vif}) thereby preventing proteasomal degradation of poly-ubiquitinated cytoplasmic APOBEC3s via that specific virus-encoded mechanism. An example of provirus transcription-triggering stimuli are the latency reactivating agents (LRAs) used here.

Another aspect of the disclosure is a method of inducing remission (and sustained remission) of HIV by administering an inhibitor of NAE1 before and/or during reactivation of latent virus by delivery of (or also potentially activation of) a latency reactivating agent (LRA) during ART. A LRA is an agent that activates an immune cell harboring HIV provirus, or HIV components, in a latent state to reactivate production of virus or viral components. The latent HIV provirus or components are well established to be present during suppressive ART. In some embodiments, adding a neddylation inhibitor (e.g. inhibitor of NAE1), such as MLN, to latency reactivating agents and suppressive ART may allow a lower level of latent virus reactivation (which may be easier to handle by natural immunity or an adjunctive immune intervention). It also minimizes infectivity of any reactivated virus, helping to deplete the reservoir of latent HIV proviruses by adding to ART's limited effect to block “replenishment” of HIV's latent reservoir by reactivated viruses. Delivery of a LRA during viremia-suppressing ART can allow persistent and latent replication-competent viral genomes that are present to be reactivated. Adjunctive MLN can substantially add to limiting reactivated virus spread, and have other effects contributing to a sustained remission after stopping ART and the NAE1 inhibitor. In embodiments, methods of administering an inhibitor of NAE1 before and/or during reactivation of latent provirus under cover of viremia-suppressing ART are described that can help deplete a reservoir of HIV or latent HIV. In embodiments, the inhibitor of NAE1 is MLN.

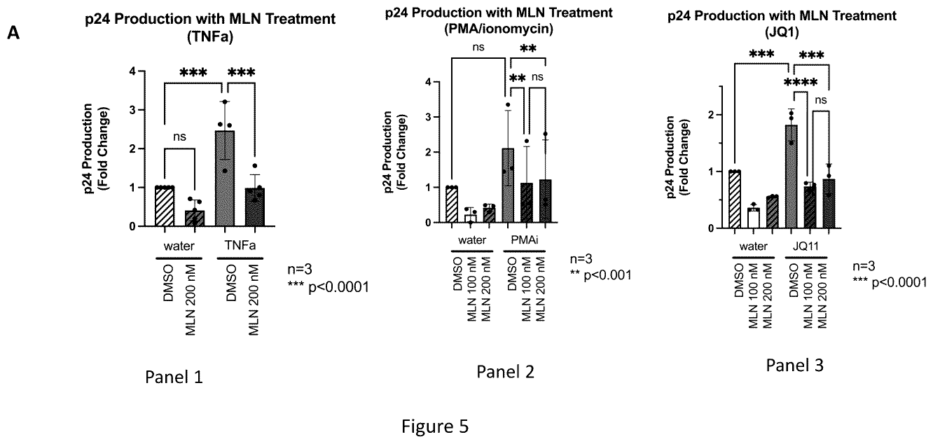

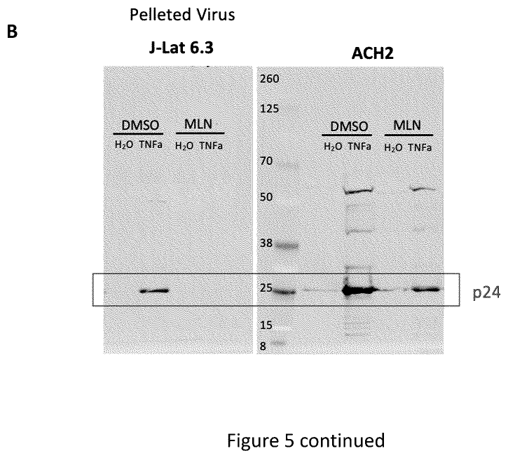

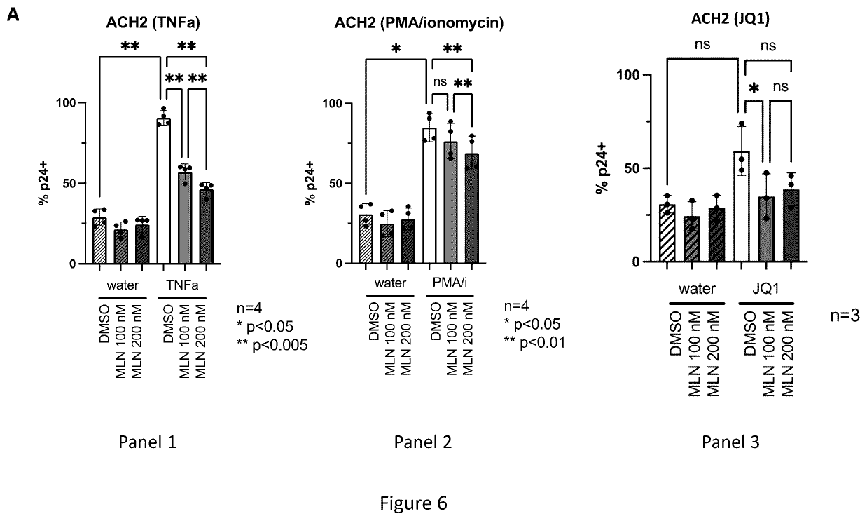

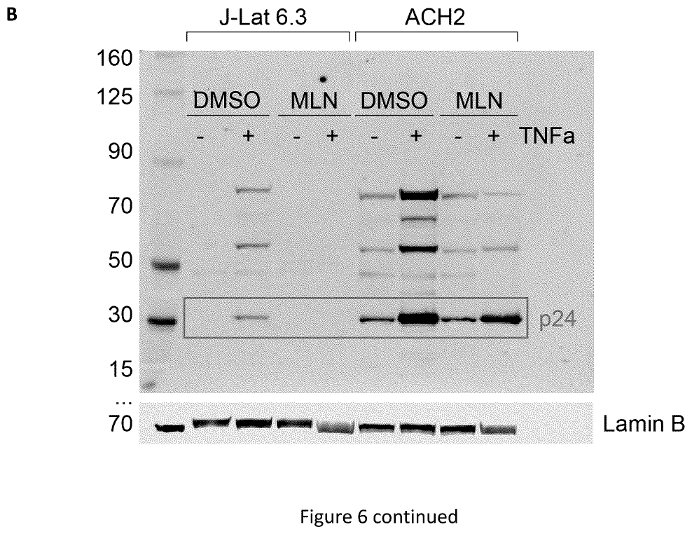

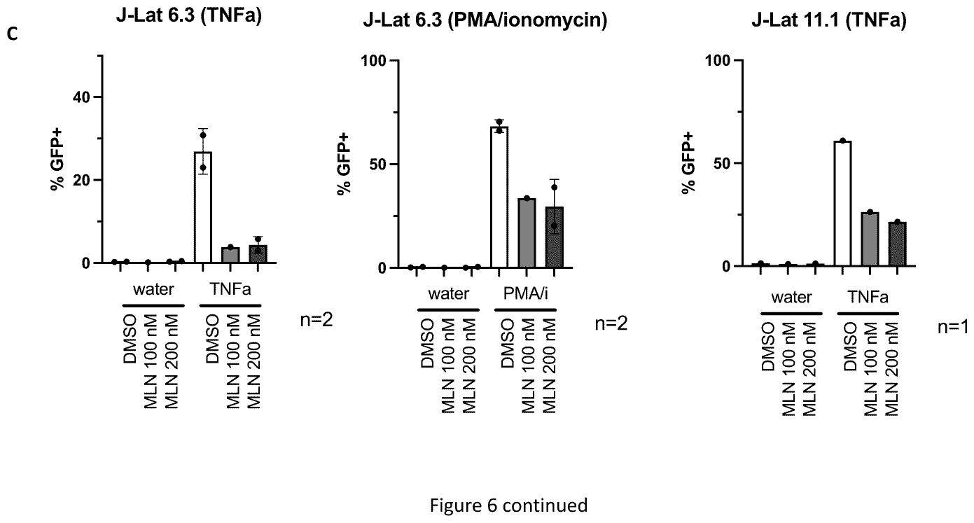

In embodiments, administering an inhibitor of NAE1 before delivery of or activation of a LRA limits virus production from reactivated latent genomes (FIGS. 5, 6). In embodiments, administering an inhibitor of NAE1 before delivery of a LRA substantially limits reactivated virus infectivity. The limitation by MLN to spread of infectious virus may be from any HIV components that manage to be produced and assembled into viruses via effects on both virion infectivity (FIG. 3) and previously established effects of MLN on cell cycling that impact target cell susceptibility to HIV. In embodiments, the methods prevent or limit both reactivation of latent virus and its spread; these combined effects can allow ART to be stopped temporarily or completely.

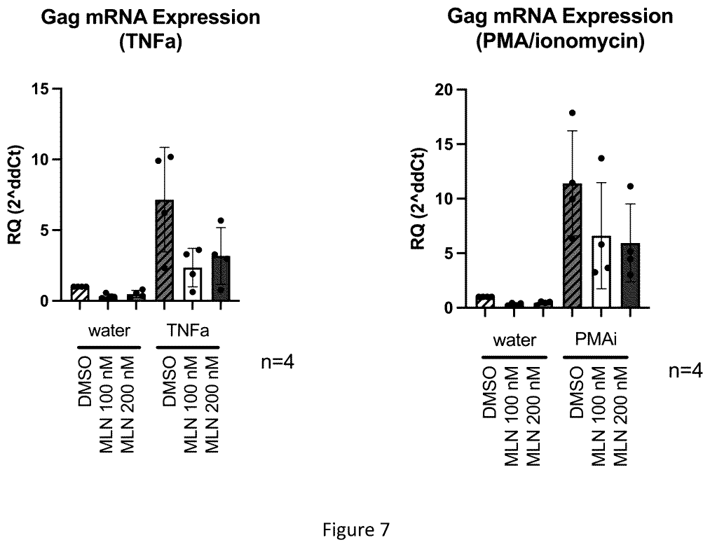

An inhibitor of NAE1 is further shown to decrease reactivated proviral transcriptional activity (FIG. 7). In embodiments, treatment with MLN decreases HIV RNA expression which decreases initial steps in provirus reactivation.

Moreover, another aspect is a method of improving treatment of HIV by administering an inhibitor NAE1 to a subject just before and after ART has stopped or ended (without administering an LRA). Administering an inhibitor of NAE1 may be for a temporary period. Use of a provirus transcription-activating LRA is not needed to reactivate latent HIV or components in this circumstance, as this occurs spontaneously within days to weeks after ART stops. In such a case, administration of an inhibitor of NAE1, such as MLN, limits virus production from reactivated latent genomes or virus. In embodiments, methods of improving HIV treatment to attain sustained remission while off medications include administering an inhibitor of NAE1 temporarily shortly before and after ART stops to decrease both latent provirus reactivation and reactivated virion spread to limit or stop recurrence of viremia after ART stops.

Another aspect is a method of minimizing production of immune-activating virus or virus components or remnants that continue to contribute to systemic inflammation even during viremia-suppressing ART by using an inhibitor of NAE1, thereby decreasing risk of developing comorbidities that are now the major causes of morbidity and mortality among ART-treated persons with HIV (or ameliorating those already occurring). ART suppression of active viral replication does not prevent ongoing immune cell activation and systemic inflammation from the immune cell recognition of HIV remnants, which can cause or accelerate the following in ART-treated patients: cardiovascular disease (heart attack, heart failure, stroke); cancers not caused by immunodeficiency (notably lung and head and neck cancers); performance-impairing cognitive decline; damage to bone, liver and kidneys. Thus, an inhibitor NAE1 may be administered during ART to decrease immune cell activation and systemic inflammation from the immune recognition of virus remnants, thereby decreasing comorbidities in persons with ART-treated HIV.

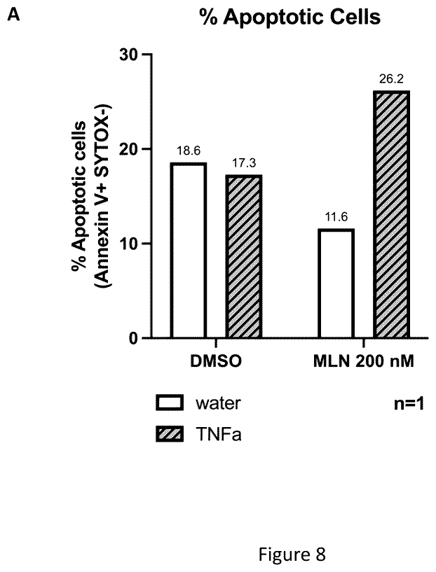

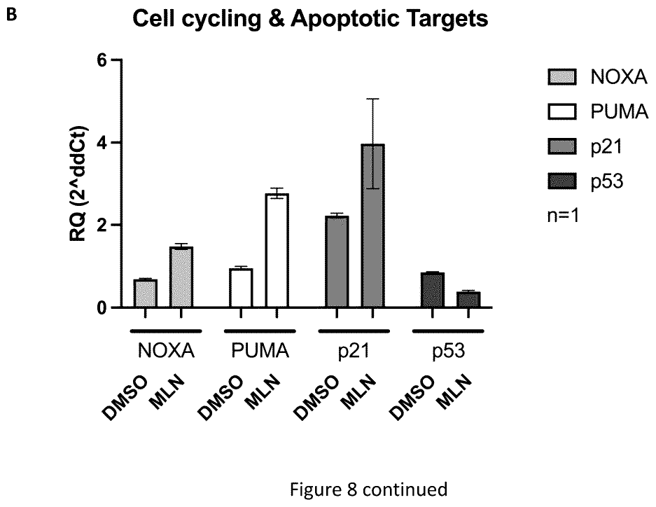

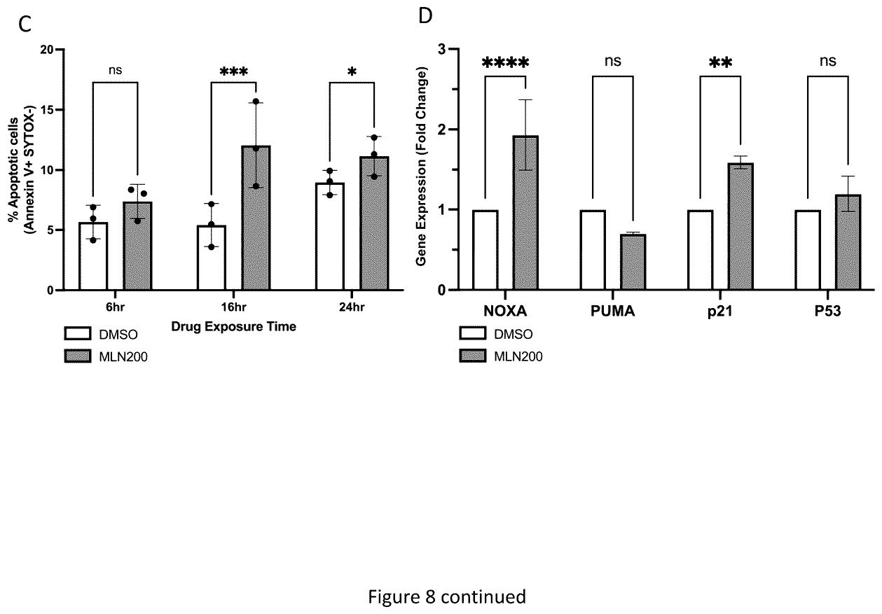

An inhibitor NAE1 is also shown to induce apoptotic cell death in cultures of cells reactivating provirus following LRA exposure (FIG. 8). Work is ongoing to test a hypothesis that apoptotic cell death is specific to (or preferential for) the cells reactivating latent provirus transcription. Thus, another aspect is use of an inhibitor NAE1 to induce apoptotic cell death in provirus-reactivating cells, potentially specifically. Such cell death (whether specific to HIV-reactivating cells or not) can prevent a reactivated provirus from returning to latency and can deplete provirus-expressing cells, either by itself or with an adjunctive intervention enhancing cytotoxic immune effector mechanisms specifically recognizing HIV. In embodiments, administering an inhibitor of NAE1 increases apoptotic cell death in cultures of cells reactivating virus ex vivo. In embodiments, administering an inhibitor of NAE1 induces the pro-apoptotic factors PUMA (p53 upregulated modulator of apoptosis) and NOXA (phorbol-12-myristate-13-acetate-induced protein 1) in both a T cell line and primary CD4 T cells (FIG. 8).

A further aspect of the disclosure is directed to a pharmaceutical composition that includes an inhibitor of NAE1 for use in the described methods. The pharmaceutical composition may comprise a therapeutically effective amount or pharmaceutically acceptable amount of the NAE1 inhibitor. The composition may also include any of the disclosed ART, LRA, or other agents used in disclosed methods. The pharmaceutical composition may also include a pharmaceutically acceptable carrier.

### EXAMPLES

Neddylation is a post-translational modification that regulates protein degradation, cellular transcription and cell cycling. In the process of neddylation, ubiquitin-like protein neural-precursor-cell-expressed developmentally down-regulated 8 (NEDD8) is conjugated to its target protein, using NEDD8 specific E1 and E2 enzymes. Because pevonedistat (formerly MLN4924, called MLN here), a NEDD8-activating enzyme inhibitor that potently inhibits neddylation, was previously reported to block Cullin RING Ligase (CRL^{Vif})-mediated degradation of the potent HIV-1 restriction factor APOBEC3G (A3G) in ex vivo, de novo HIV infections, its effect on reactivation of latent provirus was studied. Many additional effects are identified and well characterized here.

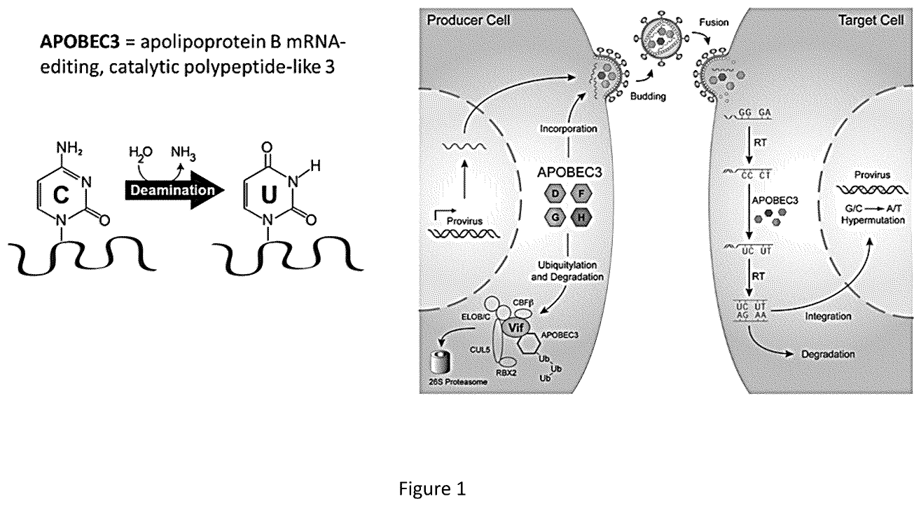

A3G is one of seven human APOBEC3 cytidine-deaminases. It strongly inhibits HIV-1 replication by mutating C to T in HIV ssDNA during reverse transcription if packaged into virions (FIG. 1). Packaging requires that A3G escape degradation in the producer cell by HIV-encoded viral infectivity factor (Vif) via its Cullin-RING ligase (CRL^{Vif}). A3G must also remain in its virion-packageable low-molecular-mass (LMM) form in the producer cell cytoplasm. However, during T cell activation to proliferate during an immune response, when cells become most susceptible to HIV infection, A3G moves in the cytoplasm to become sequestered into high-molecular-mass (HMM) ribonucleoprotein complexes from which it cannot be packaged into virions. If a T cell is reactivating previously latent provirus transcription following such signals for T cell proliferation, both HMM sequestration and degradation of LMM forms by CRL^{Vif} preclude new virions containing A3G antiviral activity. MLN can increase virion packaging of A3G (FIG. 3) and limit the shift of A3G to the non-packageable HMM complexes (FIG. 4).

In a cell line harboring a Vif+, replication-competent provirus and expressing A3G (ACH2 cells), pevonedistat treatment before exposure to several latency reactivating agents (LRAs) is shown to lead to more A3G within, and decreased infectivity of, reactivated virions (FIG. 3). Importantly, in addition, decreased reactivated HIV RNA and protein production was shown with pevonedistat exposure for short times prior to and during LRA treatment in several latently infected cell lines, whether the cells expressed A3G or not (ACH2, J-Lat 6.3, J-Lat 11.1) (FIGS. 5-7). Multiple mechanisms by which MLN treatment decreases production and spread of reactivated, infectious viruses from T cell lines harboring latent provirus are identified.

Pevonedistat (or MLN as used in this disclosure) is known (and shown here as well) to increase expression of p21, which has been shown to decrease CDK9 augmentation of transcription required to initiate transcription from the HIV promoter. MLN also is shown here to increase apoptotic cell death in cultures of T cell lines reactivating latent provirus and induces the pro-apoptotic factors PUMA and NOXA in both a cell line and primary CD4 T cells (FIG. 8). Whether these effects are also seen in primary T cells from persons living with HIV (PLWH) on effective, prolonged viremia-suppressing antiretroviral therapy (ART) is now being studied ex vivo. This work already demonstrates that broadly inhibiting neddylation can limit infectious provirus reactivation from latently infected T cells via three mechanisms: 1) reducing HIV provirus transcription and reactivated virion production (FIGS. 5-7), 2) reducing infectivity of the virions that do reactivate via increased packaging of A3G, with one mechanism for this increased packaging suggested (FIGS. 3 and 4), and 3) inducing apoptotic cell death in cultures of provirus-reactivating cells (FIG. 8). The latter effect precludes at least some of the cells reactivating provirus (and perhaps all or most, now under study) from surviving and returning to the prior state of harboring a latent provirus. These effects have the potential to overcome the problematic return to latency seen in experimental approaches limited to exposure to only LRAs under cover of viremia-suppressive ART even with adjunctive HIV-targeted, immune-enhancing interventions. Thus, inhibiting neddylation using pevonedistat warrants further study as an adjunctive HIV cure strategy because it targets multiple mechanisms contributing to reservoir depletion.

Advantages of the disclosed technology include, without limitation: using LRAs alone (or with an adjunctive immune intervention) to “shock and kill” and “purge” persistent HIV under cover of ART) may activate immune cells, further increasing risk and severity of immune activation-related, aging-associated comorbidities. The disclosed technology provides for minimization of production of immune-activating virus components, thereby minimizing risk of worsening systemic inflammation causing comorbidities that are now important causes of morbidity and mortality during effective ART. Moreover, the inhibitor may be used without using an LRA simply by stopping ART.

### Example 1. MLN Inhibits Neddylation at Concentrations Used Here

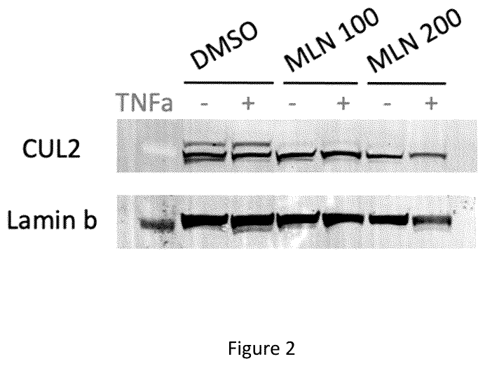

FIG. 2 shows that MLN inhibits Cullin 2 (CUL2) neddylation at 100 mM and 200 mM in ACH2 cells. Data not shown indicate similar effects of MLN on CUL2 neddylation in CEM, Jurkat and J-Lat cells.

### Example 2. MLN Decreases the Amount of Virus Produced after Latency Reactivation

MLN decreases reactivation of latent provirus, as shown in VIF+, replication-competent provirus, from the latently infected ACH2 T cell line based on HIV capsid p24 amounts in cell culture supernatant fluid and intracellularly after 3 different LRAs: tumor necrosis factor alpha (TNFa) at 10 ng/mL; PMA/ionomycin (PMAi) at 405 pM phorbol 12-myristate 13-acetate (PMA) and 6.7 nM ionomycin (eBiosciences™ stimulation cocktail); and JQ1 at 100 nM (FIGS. 5 and 6). Culture supernatant p24 expression is reduced in MLN pretreated, reactivated, ACH2 cells showing virus production is significantly reduced (FIG. 5A). Immunoblotting of pelleted supernatant from reactivated ACH2 and J-Lat 6.3 cells shows reduced extracellular p24 capsid with MLN treatment compared to control (FIG. 5B). MLN reduces intracellular p24 production from ACH2 1 cells treated with TNFa, PMAi, and JQ1, compared to DMSO control (FIG. 6A). MLN also reduced intracellular GFP expression from J-Lat 6.3 treated with both TNFa and PMAi as well as J-Lat 11.1 cells treated with TNFa (FIG. 6C). HIV promoter-driven RNA is also shown to be decreased by MLN before/during TNFa or PMAi (FIG. 7). FIG. 7 shows HIV gag RNA expression is decreased with MLN treatment (e.g., decrease in proviral transcriptional activity) in ACH2 cells as measured by RT-qPCR 48 hours after reactivation. ACH2 cells were pretreated for 6 hours with DMSO, or MLN at 100 mM and 200 mM, and then reactivated with 10 ng/mL TNFa or PMA/ionomycin.

### Example 3. MLN Reduces Infectivity of the Smaller Amount of Reactivated Virions, Increases their A3G Content and Limits Shift of A3G Intracellularly into HMM Complexes

Virus produced from ACH2 cells treated with MLN before/during exposure to 3 different LRAs are less infectious compared to those released from untreated (DMSO control) cells (FIG. 3A). Virus infectivity was determined by infecting TZM-bl reporter cell line with p24-equivalent amounts of culture supernatant fluid (e.g., normalized to the same p24 amount, as a surrogate for inoculating the same number of virions) from cells pretreated with MLN or DMSO control and reactivated by the 3 LRAs). Supernatant infectivity was measured via luciferase activity in the form of Relative Fluorescence Units (RFU) where higher RFU signifies greater infectivity. Average RFU values were normalized to the control condition (LRA alone without MLN treatment) such that maximum infectivity seen in that control condition is set to 100%.

FIG. 3B shows MLN pretreatment of ACH2 cells before TNFa reactivation increases the amount of A3G incorporated into budding virions, explaining decreased infectivity after reactivation shown in FIG. 3A. Virions from ACH2 cell supernatant were pelleted over a 20% sucrose cushion. Immunoblotting of viral pellets reveals A3G inside the virions is increased with MLN treatment (left panel, Panel 1, shows one representative immunoblot image). A3G signal (black bars) and p24 signal (grey bars) from n=3 experiments are quantified on the right (Panel 2) in FIG. 3B.

MLN was shown to limit shift of A3G into sequestered/non-packageable HMM complexes in H9 cells and primary CD4+CD45RO+ memory T cells isolated from healthy donors and then T cell receptor stimulated with anti-CD3/28 (FIG. 4).

### Example 4. MLN Induces Apoptotic Cell Death in Reactivated Latently Infected T Cells

FIG. 8 shows MLN induces apoptosis in reactivated latently infected ACH2 T cells. Annexin V staining of ACH2 cells (FIG. 8A) and CD4+ T cells (uninfected) treated with MLN (FIG. 8C at 16 hours) shows an increase in apoptosis-mediated cell death in MLN treated cells. ACH2 cells were treated with 200 nM MLN for 6 hours before and then during reactivation with 10 ng/mL TNFa. 16 hours post-TNFa addition, cells were collected and percentage of apoptotic cells increased with MLN treatment upon reactivation (FIG. 8A). RT-qPCR for pro-apoptotic targets (PUMA, NOXA) and proliferation/cell cycle indicators (p21, HIF1a) shows an increase in pro-apoptotic gene expression (PUMA, NOXA) and p21 expression in ACH2 (FIG. 8B) and in CD4+ T cells (FIG. 8C). Expression of p53 RNA did not significantly increase in either ACH2 or CD4+ T cells (FIG. 8A, C).

### Other Definitions

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The term “patient” or “subject” refers to all members of the animal kingdom prone to or suffering from the indicated disease or disorder. The subject may be a mammal, e.g., a human or non-human.

The term “effective amount” as used herein generally refers to a concentration or amount of a compound, material, or composition, as described herein, that is effective to achieve a particular biological result.

The term “therapeutically effective amount” includes the amount of the inhibitor or composition of the present disclosure or a pharmaceutically acceptable version thereof, that when administered, induces a positive modification in the disease or disorder to be treated (e.g., latentcy or remission), or is sufficient to prevent development or progression of the disease or disorder, or alleviate to some extent, one or more of the symptoms of the disease or disorder being treated in a subject, or which simply kills or inhibits the growth of diseased (e.g., HIV or provirus) cells.].

“Pharmaceutically acceptable” refers to that which is useful in preparing a pharmaceutical composition that is generally safe, non-toxic and neither biologically nor otherwise undesirable and includes that which is acceptable for use in a subject, including human pharmaceutical use.

The term “pharmaceutically acceptable carrier,” as well-known in the art, refers to a pharmaceutically acceptable material, medium, or composition that is suitable for use as a carrier or vehicle to introduce, deliver, or administer a compound or compositions, such as the NAE1, to a subject.

Suitable carriers may include, for example, liquids (both aqueous and non-aqueous alike, and combinations thereof), solids, encapsulating materials, gases, and combinations thereof (e.g., semi-solids), that function to carry, transport, or deliver a material, compound, composition or the like to a subject, which may include to a cell, tissue, or organ, or portion of the body.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method of treating a human immunodeficiency virus (HIV) infection in a subject comprising administering to the subject an inhibitor of neddylation activation enzyme neural-precursor-cell-expressed developmentally down-regulated 8 (NEDD8) E1 (NAE1).

2. The method of claim 1, wherein the inhibitor of NAE1 is pevonedistat.

3. The method of claim 1, wherein the inhibitor of NAE1 decreases production of infectious HIV from latent HIV provirus or other HIV latency components in T cells.

4. The method of claim 1, wherein HIV-1 restriction factor APOBEC3G (A3G) is increased within reactivated HIV following exposure to the inhibitor of NAE1.

5. The method of claim 1, wherein the inhibitor of NAE1 decreases infectivity of reactivated HIV.

6. The method of claim 1, wherein the inhibitor of NAE1 reduces HIV provirus transcription and HIV viral protein production.

7. The method of claim 1, wherein the inhibitor of NAE1 is administered prior to and during short-term exposure to a latency reactivating agent (LRA).

8. The method of claim 1, wherein the inhibitor of NAE1 is administered to the subject before and for a defined time period after stopping antiretroviral therapy (ART), wherein the inhibitor of NAE1 prevents or limits reactivation of HIV production after stopping ART to a magnitude that prevents or limits recurrence of viremia after stopping ART in the subject.

9. The method of claim 1, wherein the inhibitor of NAE1 prevents or limits immune cell activation and systemic inflammation from HIV during viremia-suppressing ART.

10. The method of claim 9, wherein the method decreases the risk of acquisition or progression of HIV-associated comorbidities.

11. The method of claim 10, wherein the comorbidities are one or more of cardiovascular disease, cancer not caused by immunodeficiency, performance-impairing cognitive decline, damage to bone, damage to liver, and damage to kidneys.

12. The method of claim 11, wherein the cardiovascular disease is heart attack, heart failure, or stroke.

13. The method of claim 11, wherein the cancer not caused by immunodeficiency is lung, head, or neck cancer.

14. The method of claim 8, wherein the inhibitor of NAE1 is administered to the subject before and for a defined time period after stopping ART in the absence of any LRA exposure.
